(19) United States
(12) Patent Application Publication
(10) Pub. No.: US 2005/0064481 A1
(43) Pub. Date: Mar. 24, 2005

(54) Method for reducing background contamination

(76) Inventor: Christian Korfhage, Langenfeld (DE)

Correspondence Address: LEON R. YANKWICH;YANKWICH & ASSOCIATES, 201 BROADWAY, CAMBRIDGE, MA, 02139, US

(21) Appl. No.: 10/918,975

(22) Filed: Aug. 16, 2004

Related U.S. Application Data
(63) Continuation-in-part of application No. PCT/EP03/01590, filed on Feb. 17, 2003.

(30) Foreign Application Priority Data
Feb. 15, 2002 (DE) 102 06 616.7

(51) Int. Cl. C12Q 1/68

(52) U.S. Cl. 435/6

(57) ABSTRACT

The present invention relates to a method for reducing background signal in a biomolecule labelling reaction.

### CROSS-REFERENCE TO RELATED APPLICATIONS

This application is a continuation-in-part of copending international (PCT) application No. PCT/EP03/01590, filed Feb. 17, 2003, designating the United States, which application claims priority to German Patent Appln. No. 102 06 616.7, filed Feb. 15, 2002.

### FIELD OF THE INVENTION

The invention relates to a method for reducing background contamination when carrying out labelling reactions on biomolecules, preferably on biopolymers such as peptides, proteins or nucleic acids with labelling substances.

### BACKGROUND OF THE INVENTION

In the prior art, labelling reactions have become well established for quantifying or identifying substances. Thus, for example, nucleic acids may be labelled with modified nucleotides. In other cases labelled substances are used as codes or sensors for identifying other molecules or for monitoring processes.

In order to obtain pure labelled substances, correspondingly labelled molecules are used as substrate in these labelling reactions. Non-radioactive modifying groups are incorporated by enzymatic, photochemical or chemical reactions. Radioactive isotopes, on the other hand, are largely incorporated by enzymatic reactions. The labelling positions and type of labelling differ in the different processes depending on whether radioactive isotopes or non-radioactive reporter groups are used.

By enzymatic incorporation of labelled nucleotides, nucleic acids (DNA, RNA) of all kinds of lengths can be labelled. In this way it is possible to produce probes which have a high labelling density and high sensitivity. In the enzymatic labelling reactions, either 5′-labelled primers (PCR labelling) or labelled nucleotides are used instead of non-labelled nucleotides or a combination of both methods is used.

The disadvantage inherent in all these labelling techniques is that the labelled product has background contamination which constitutes interference. This problem is the starting point of the present invention.

A number of purification methods for eliminating unwanted contaminants from the desired labelled product are known from the prior art. However, these often have the drawback that when the labelled substances are used contamination can still be detected.

The aim of the present invention is therefore to provide a process which makes it possible to discriminate similar properties in the labelled biomolecules. Thus, the aim of the present invention is to provide a substance preparation with the lowest possible background contamination which is easy to carry out.

In numerous molecular biological applications, nucleic acid fragments, oligonucleotides, proteins and peptides are labelled, for example, with radioactive compounds or with dyes. The method used for labelling is determined, however, by the length of the nucleic acid fragments, by the experimental requirements and the detection sensitivity, for example. The labelling reaction may be catalysed using polymerases such as DNA or RNA polymerases or reverse transcriptases.

The radioactive labelling is carried out by incorporating unstable isotopes or substances which contain unstable isotopes or by replacing stable natural isotopes with unstable isotopes [F. Lottspeich and H. Zorbas (Editors), Bioanalytik, Spektrum Akademischer Verlag, Heidelberg, 1998]. The chemical structure moreover, is unaffected by this exchange of isotopes, which means that the labelled molecules have the same physico chemical properties as the natural substances. This means that the reaction conditions for the enzymatic incorporation of labelled nucleotides in hybridisation probes need not be changed. In the case of labelling with ³²P or ³³P-phosphate, which is most common, the exchange position is either the α- or γ- position of the phosphate groups in 2′-deoxyribo, 3′-deoxyribo or 2′-ribonucleotides.

In the case of ³⁵S the exchange is made for an oxygen atom of the α-phosphate.

Labelling with ¹²⁵I is carried out at the C-5-position of the cytosine.

In contrast to isotope labelling, when using non-radioactive molecules to modify probes by inserting the modifying group the chemical structure of the labelled nucleotides is altered. The result of this is, for example, that the reaction conditions for enzymatic incorporation have to be adapted to the different substrate properties. To protect against non-specific interactions, during blot hybridisations, the membrane surface and, in in situ hybridisations, the cell or tissue surface have to be saturated with suitable blocking reagents.

Labelled nucleic acids (DNA and RNA) are used as reagents or samples in molecular biological cloning. In general, the marking reagent may be covalently bound to the substance which is to be labelled or may be non-covalently associated therewith.

Labelled fragments of cloned DNA and/or oligonucleotides of defined size are used as a reagent for chemical and enzymatic sequencing, for nuclease S1 analysis of RNA and in so-called band shift experiments. On the other hand, labelled nucleic acid samples are required in hybridisation techniques for locating and binding DNA and RNA of complementary sequences. These techniques include colony and plaque hybridisation, Southern and Northern analysis, in situ hybridisation and sequencing by hybridisation. In the cases described the success of the introduction of a label into DNA or RNA is dependent on the method used, such as e.g. end labelling, random priming, Nick translation, in vitro transcription and variations of polymerase chain reaction (PCR), etc.

As an explanation first of all some methods of labelling nucleic acids known from the prior art will be described. Within the scope of the present invention modifications or variations of these methods and totally different methods may be used.

5′-phosphorylation of DNA

Using [γ-³²P]ATP, radioactive labelling of single-strand hybridisation probes or DNA is carried out for sequencing according to Maxam-Gilbert. The γ-phosphate group of the ATP is transferred by the enzyme T4-polynucleotide kinase to the 5′-terminal hydroxyl group of the oligodeoxynucleosidetriphosphate or DNA.

Terminal Transferase Reaction

DNA oligonucleotides can be enzymatically labelled by terminal transferase by matrix-independent attachment of labelled dNTPs (tailing). If mixtures of labelled and unlabelled dNTPs are used single strand chains (tails) are formed in a matrix-independent reaction, these tails carrying several labels. When labelled Cordyceptintriphosphate (3′-dATP) or 2′,3′-ddNTPs are used only a single labelled nucleotide is attached as the reduced 3′ position cannot be further extended.

3′-End Labelling of DNA

DNA with 5′-projecting ends can be radioactively labelled by a reaction of filling the Klenow fragment of E. coli DNA polymerase I at one or both 3′-ends. This is done using the [α-³²P]deoxynucleosidetrisphosphates which are complementary to the first base of the 5′ single strand ends.

Radioactive Labelling of DNA by Nick-Translation

In Nick translation, the E. coli DNA Polymerase Holoenzyme is used in addition to small amounts of pancreatic DNase I. The background to this is that in this method 5′→3′-exonuclease activity is required in addition to the polymerase activity. First of the DNase catalyses the formation of single strand breaks (nicks). To ensure that no further splits occur a precisely adjusted low DNase concentration is required. The 3′ and 5′-ends flanking the nick act as a substrate for the 5′→3′ polymerase activity and the 5′→3′-exonuclease activity. The 5′→3′-exonuclease activity is responsible for the successive breakdown of the 5′-phosphorylated nucleotides, whereas gaps produced synchronously by the 5′→3′ polymerase activity are filled in again with new labelled nucleotides. As a result the Nick migrates in the direction 5′→3′ (nick translation). Accordingly this is DNA replacement synthesis, in which the yields remain below 100%, in contrast to random priming synthesis (see below) for the reaction-based reasons described above.

Labelling of DNA Fragments by “Random Priming”

In the random priming procedure first of all double strands DNA is denatured. The re-hybridisation of both strands is prevented by cooling to low temperatures and adding high concentrations of the primers. The primers constitute a mixture of all possible hexanucleotides (random primers) so that, viewed statistically, every target sequence is covered and hybridisation can take place at any desired location. The Klenow fragment, the large subtilisin fragment of the DNA-Polymerase-Holoenzyme, extends the primer in a matrix-dependent reaction. In the reaction of elongation, non-labelled dNTPs and hapten-modified dUTPs are incorporated. As the template strands are replicated, new synthesis takes place. In this way, high probe yields can be obtained with more than 100% of the template DNA used, by strand displacement. As statistically more primers bind per target, only partial sequences are replicated for each primer elongation; as a result a mixture of different probe lengths is obtained, except that the partial sequences are all target-specific and carry homogenous labelling. With digoxigenin-labelled probes produced by the random priming method, high detection sensitivities can be achieved in the subpicogram range.

Labelling of RNA by In Vitro Transcription

Labelled RNA probes of a high specific activity can be produced by in vitro transcription of cloned DNA fragments. This requires suitable promoters which are found for example in cloning vectors (transcription vectors) such as vectors of the Ribo Gemini series (pGEM-3 or pGEM-4). RNA samples of opposite orientation (e.g. sense and anti-sense) can be produced by these vectors with the corresponding RNA polymerase by in vitro transcription reactions. For this the vector must be linearized downstream of the sequence which is to be amplified so as not to produce any RNA fragments which run around the entire vector (run-around transcripts). In this way only the desired cloned sequence is labelled with [α-³²P]-nucleotides (ATP or CTP).

Non-Radioactive Labelling of Nucleic Acids

In the introduction of non-radioactively labelled substances, chemical groups or components which are not a part of nucleic acids conjugate with the sample by enzymatic or other chemical reactions. After hybridisation with nucleic acid, suitable indicator systems or detection systems detect the modified groups of the sample.

The first non-radioactive methods were developed back in 1980 and are based on labelling nucleic acid samples with dinotrophenol, bromodeoxyuridin and biotin.

The biotinylated samples are detected after hybridisation by interaction with Streptavidin, which is often conjugated with alkaline phosphatase as reporter enzyme, by means of the enzymatic activity of the phosphatase.

Enzymatic Methods

The homogeneous DNA labelling is achieved either by random priming with the large fragment of E. coli DNA Polymerase I (Klenow Enzyme), Nick translation of E. coli DNA Polymerase I (Kornberg Enzymes) or by PCT Amplification with Taq-Polymerase. The labelling densities amount to 1 label per 25 to 36 base pairs. Oligonucleotides can be enzymatically labelled using the terminal transferase reaction; depending on the substrate 1-5 labels are attached per oligonucleotide.

Photochemical Labelling

Nucleic acids can be labelled with biotin and digoxigenin (DIG) which are linked via a nitrophenylazido group. Irradiation of the nitrophenylazido group with UV light produces a photochemical reaction. Reactive nitrenes are cleaved at the same time.

Detection of Non-Radioactively Labelled Samples after Hybridisation

Chemoluminescence

Chemoluminescence is a fast and sensitive parameter for detecting DNA. It uses antibodies which specifically bind the labels introduced into the DNA, e.g. biotin, fluorescene or dioxigenin, and are coupled for example to horseradish peroxidase (HRP) or alkaline phosphatase. Both enzymes can be used in reactions in which light is emitted or a colour change takes place.

Fluorescent Labelling

Fluorescent labelling with fluorophores is used particularly in polypeptides or small proteins, especially those which cannot be detected with Coomassie-Blue and/or with silver staining. Fluorescent labelling may be used as an alternative to staining techniques. In nucleic acids fluorophore labelling is widely used in hybridisation systems such as microarrays.

As already mentioned previously the aim of the present invention is to provide a process in which the background contamination after the labelling reaction is reduced.

In addition to the radioactive labels or reporter groups already mentioned—of which ³H, ¹⁴C, ³²P, ³³P, ³⁵S or ¹²⁵I, or a mixture thereof are preferred—it is also possible according to the invention to use non-radioactive labels. Reporter groups of this kind are known from the prior art [C. Kessler, Nonradioactive Analysis of Biomolecules, J. Biotechnol. 35 (1994) 165]; of these the following are preferred:

Fluorescent markers such as, for example, markers for direct fluorescence: fluorescein (FITC, FLUOS), cyanines, Alexa-fluorophores, rhodamine (RHODOS, RESOS, RESIAC), hydroxycoumarin (AMCA), benzofuran, Texas-Red, biman, ethidium/Tb³⁺ or mixtures thereof.

Fluorescent markers for time-released fluorescence such as, e.g., a complex, a micelle or a chelate comprising a lanthanoid, preferably Eu³⁺ and/or Tb³⁺.

Fluorescent labels for fluorescent energy transfer such as fluorecein: rhodamine.

Luminescent markers for chemoluminescence such as for example (ISO-) luminol derivatives or acridin esters.

Luminescent markers for electroluminescence such as e.g. Ru²⁺-(2,2′-bipyridyl)₃- complexes.

Luminescent labels for luminescent energy transfer such as e.g. rhodamine: luminol.

So-called metal markers, such as e.g. metal-labelled, particularly Au- and Ag-labelled antibodies.

Enzyme markers for direct enzyme coupling such as, for example, alkaline phosphatase (AP), horseradish peroxidase (POD), microperoxidase, β-galactosidase, urease, glucose-oxidase, glucose-6-phosphate-dehydrogenase, hexokinase, bacterial luciferase, glow-worm luciferase or mixtures thereof.

Enzyme markers for enzyme substrate transfer such as for example glucose-oxidase: horseradish peroxidase.

Enzyme markers for enzyme complementation such as for example inactive β-galactosidase:a-peptide.

Polymeric markers such as for example latex dye particles or polyethyleneimine.

In all the processes mentioned above the objective is to avoid a strong background signal (herein also referred to as ‘noise’) which limits the sensitivity and dynamics of the measurements.

In general, the present invention provides a method for reducing the background signal in a biomolecule labelling reaction comprising the steps of:
a) reacting the biomolecules with a labelling substance in an aqueous solution,
b) adding a non-labelled substance to the reaction mixture of step a),
c) following step b), separating the labelled biomolecule.

The term ‘labelling substance’ as used in the present invention refers to any detectable label or any carrier of a detectable label which can be introduced into or can be associated with a biomolecule. A carrier of a detectable label is for example, but is not limited to, a nucleotide coupled to a detectable label or a nucleotide analog coupled to a detectable label or an amino acid coupled to a detectable label, etc. Such carriers of a detectable label are well known to a person skilled in the art. the detectable label is preferably covalently bound to the carrier. Non-limiting examples of labelling substances are broadly discussed above. In the terms of the present invention the labelling substance is understood as one of the educts in the labelling reaction. In general, the term ‘educt’ is well defined in the art and refers to a starting material in a chemical reaction. The educt is, thus, in general understood as the opposite of a product.

Generally, the labelling substance may be covalently bound to the biomolecule which is to be labelled (e.g. nucleic acids, peptides, oligopeptides, proteins or other biomolecules) or may be non-covalently associated therewith.

The term ‘biomolecule’ as used in the present invention refers to molecules selected from the group of nucleic acid, nucleic acid analog, e.g. LNA, PNA or the like, and protein. In a preferred embodiment, the nucleic acid is DNA and/or RNA and the protein is a biomolecule comprising more than one amino acid, wherein at least two of said amino acids are coupled via a peptide bond. Nucleic acid analogs are well known to the artisan.

According to the invention the reduction in background signal is achieved by a process in which, prior to the purification of the labelled biomolecule, preferably before the end of the labelling reaction, most preferably in the last third and most particularly preferably immediately prior to the end of the labelling reaction, a non-labelled substance is added to the reaction mixture of step a).

The term ‘non-labelled substance’ as used in the present invention refers to a substance which is preferably chemically, physically or structurally related to the labelling substance. More preferably the non-labelled substance is a non-labelled derivative of the labelling substance, for example, but not limited to, nucleotide analogs or non-naturally occurring nucleotides or non-naturally occurring amino acids, or the like. The meaning of the term ‘derivative’ as used in the present invention is obvious to a person skilled in the art. Any suitable nucleotide derivative or amino acid derivative (depending on the biomolecule to be labelled) can be utilized in the present invention. These derivatives are well known to the artisan. Most preferably, the non-labelled substance is identical to the educt, i.e. identical to the utilized labelling substance, apart from the actual labelling.

Preferably the ratio of concentration of the non-labelled substance to the labelling substance in the reaction mixture formed in step b) is in the range from 1:1 to 1000:1, most preferably in the range from 10:1 to 100:1.

The addition of the non-labelled substance (step b)) is followed by a purification process (step c)) known from the art. This may be any suitable process which leads to purification of the labelled biomolecule. Such purification processes are well known to the artisan. For example, nucleic acids are often purified via a chromatographic process, e.g. by passing the aqueous solution comprising the nucleic acids through a column comprising a filter material with a silica surface under condition whereby the nucleic acids bind to the silica surface but contaminants such as monomeric nucleotides (e.g. unreacted labelled nucleotides (i.e. the labelling substance) do not bind to the silica surface.

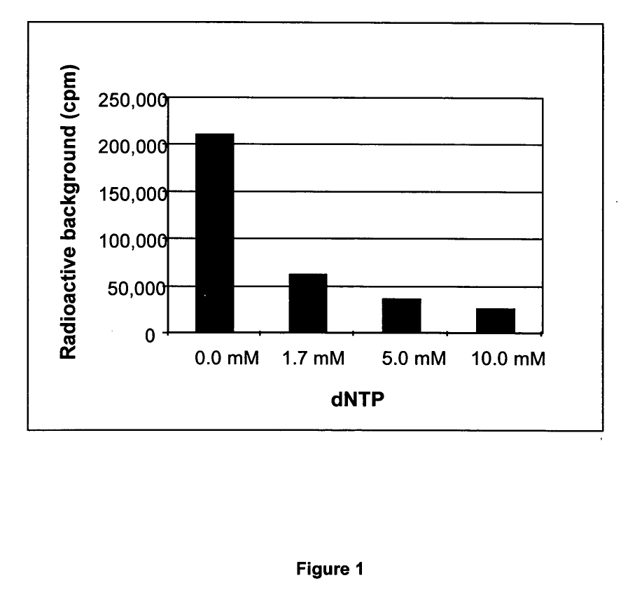

FIG. 1 shows the reduction in noise after the addition of different amounts of non-labelled substance in the labelling reaction described in Example 1.

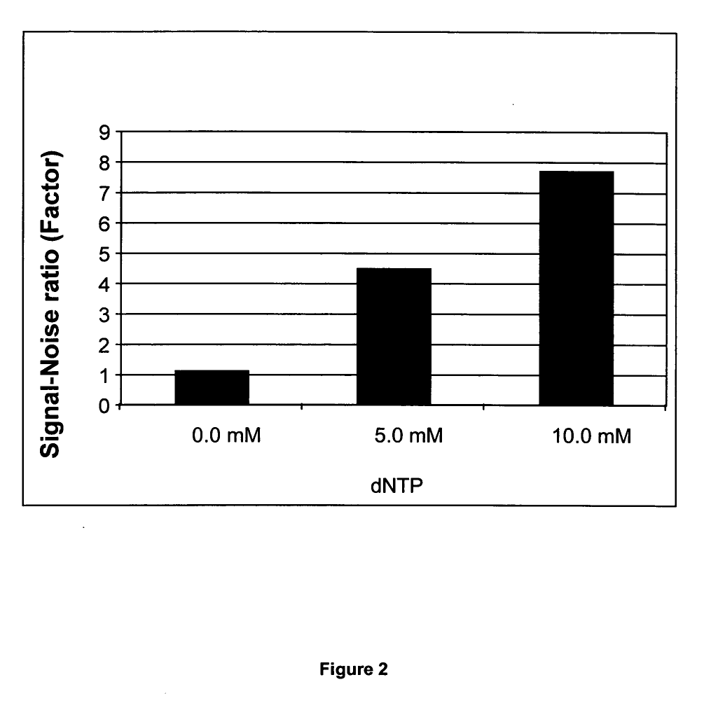

FIG. 2 shows the signal to noise ratio after the addition of different amounts of non-labelled substance in the labelling reaction described in Example 2.

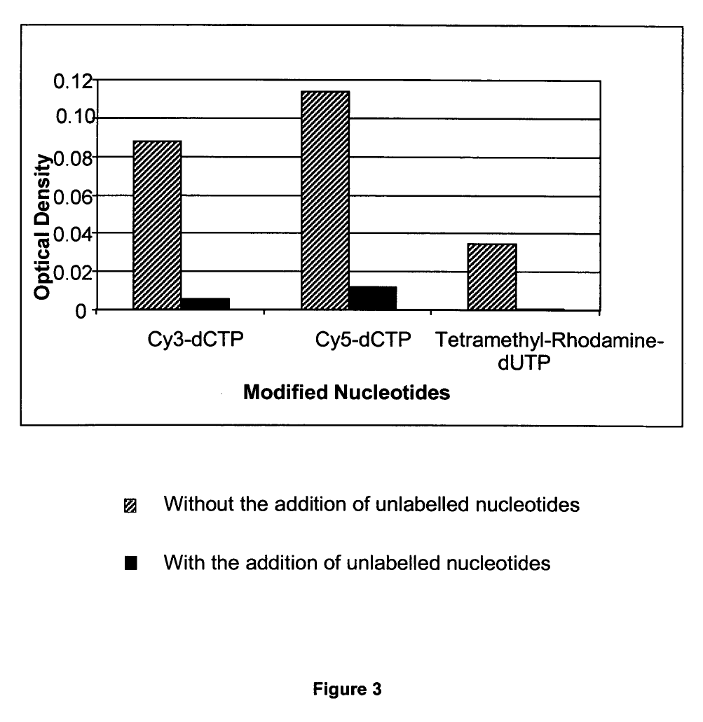

FIG. 3 graphically shows the reduction in noise after the addition of non-labelled substance in the labelling reaction described in Example 3.

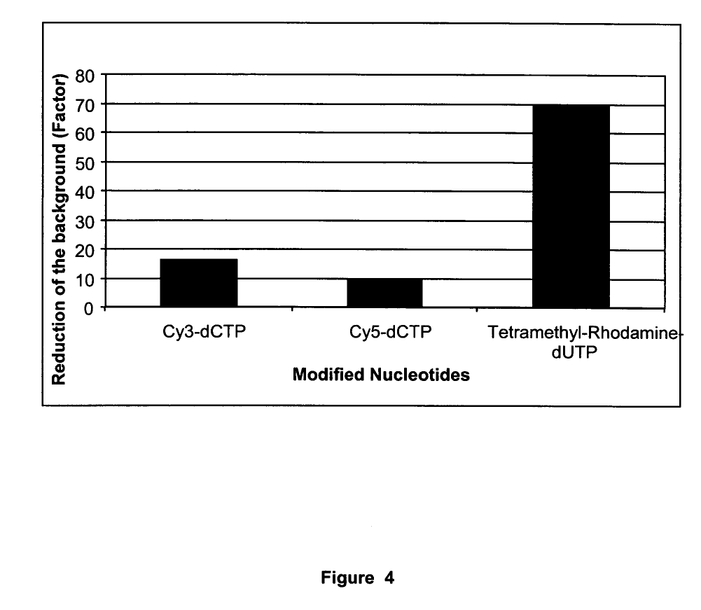

FIG. 4 shows a 70-fold reduction in the background for Example 3.

Other details of the invention are explained in the Examples.

### EXAMPLES

### Example 1

### Radioactive Labelling

In this experiment the background contamination of the radioactive nucleotides was measured after purification.

10 μCi of ³²P-dCTP were incubated together with 1 μg poly(A)-RNA, standard buffer, which buffers in a pH range of from 7 to 10 (for example commercially available RT buffer, Qiagen, D-40724 Hilden), 0.1 mM (mmol/L) of dNTP, 10 U of RNase inhibitor (Promega) and 1 μM of oligo-dT 15.

During this incubation no radioactively labelled nucleotides were incorporated as no enzymes were added. Then the mixture was incubated for 1 h at 37° C. After incubation 10 μl of a mixture which contains non-labelled nucleotides of different concentrations was added to the mixture in different reaction preparations. A reaction mixture to which 10 μl of water (0 mM dNTP) has been added was used as the control mixture.

These nucleic acid solutions were purified by a silica purification step (e.g. “QiaQuick”, Qiagen, D-40724 Hilden). The RNA bound to the silica membrane during the purification process. The eluate thus obtained, which should contain no free radioactively labelled nucleotides, only purified RNA, was measured.

A continuous reduction in the background contamination was detected after the addition of a non-labelled dNTP solution comprising 1.7 mM dNTP, 5 mM dNTP or up to 10 mM dNTP (see FIG. 1).

### Example 2

### Radioactive Labelling

In this experiment the signal to noise ratio of incorporated labelling substance to non-incorporated labelling substance was measured in comparative reactions.

10 μCi of ³²P-dCTP were incubated together with 1 μg poly(A)-RNA, standard buffer, which buffers in a pH range of from 7 to 10 (for example RT buffer, Qiagen, D-40724 Hilden), 0.1 mM of dNTP, 10 U of RNase inhibitor (Promega) and 1 μM of oligo-dT 15.

Some of the reaction mixtures contained Omniscript Reverse Transcriptase (Qiagen, D-40724 Hilden), while the other reaction mixtures did not contain any enzyme for the incorporation of radioactively-labelled nucleotides and thus act as a background control. These mixtures were incubated for 1 h at 37° C. and then supplemented with 10 μl of a mixture which contained non-labelled nucleotides of different concentrations, in different reaction mixtures. 10 μl of water (0 mM dNTP) were added to one reaction mixture. This acted as the control mixture.

The nucleic acid solutions were purified by a silica purification step (e.g. “QiaQuick”, Qiagen, D-40724 Hilden). The RNA and radioactively labelled cDNA bound to the silica membrane (commercially obtainable from Qiagen, D-40724 Hilden) during the purification process. In the control reaction the RNA bound to the silica membrane. The eluate, which should contain no free radioactively labelled nucleotides, but should contain purified RNA or RNA/radioactively labelled cDNA, was measured.

The signal to noise ratio in the eluate of incorporated labelling substance to non-incorporated labelling substance in comparative reactions was calculated.

When non-labelled nucleotides were added at the end of the reaction but before the purification the signal to noise ratio increased 8-fold (see FIG. 2).

### Example 3

### Fluorescent Labelling

In this experiment the background contamination of nucleotides labelled with fluorophores was measured after purification.

0.1 mM of fluorophore-labelled nucleotides were incubated together with 0.4 μg DNA and 0.1 mM dNTP in water. Fluorophore-labelled nucleotides could not be incorporated as no enzymes were added. These mixtures were briefly incubated and then supplemented with 10 μl of a mixture which contained non-labelled nucleotides (10 mM), in different reaction mixtures. 10 μl water (0 mM dNTP) were added to one reaction mixture. This was used as the control mixture.

All the mixtures were purified by a silica purification step (e.g. “QiaQuick”, Qiagen, D-40724 Hilden). The DNA bound to the silica membrane during the purification process. The optical density of the eluate was measured under standard conditions in the photometer (see FIG. 3).

If, at the end of the incubation but before the nucleic acid purification, non-labelled nucleotides were added to the mixture, the background contamination was reduced by up to 70-fold (see FIG. 4).

1. A method for reducing the background signal in a biomolecule labelling reaction comprising:
a) reacting the biomolecules with a labelling substance in an aqueous solution,
b) adding a non-labelled substance to the reaction mixture of step a),
c) following step b), separating the labelled biomolecule.

2. The method according to claim 1, characterized in that the biomolecule is selected from the group consisting of nucleic acids, nucleic acid analogs, and proteins.

3. The method according to claim 2, characterized in that the nucleic acid is DNA and/or RNA.

4. The method according to claim 2, characterized in that the protein is a biomolecule comprising more than one amino acid, and wherein at least two of said amino acids are coupled via a peptide bond.

5. The method according to claim 1, characterized in that the labelling substance is a detectable label or a carrier of a detectable label which can be introduced into or can be associated with said biomolecule.

6. The method according to claim 5, characterized in that the detectable label is a radioactive isotope.

7. The method according to claim 6, characterized in that the radioactive isotope is selected from the group consisting of ³H, ¹⁴C, ³²P, ³³P, ³⁵S and ¹²⁵I or a mixture thereof.

8. The method according to claim 5, characterized in that the detectable label is a fluorescent label.

9. The method according to claim 8, characterized in that the fluorescent label is selected from the group consisting of fluorescein (FITC, FLUOS), rhodamine (RHODOS, RESOS, RESIAC), hydroxycoumarin (AMCA), benzofuran, Texas red, bimane and ethidium/Tb³⁺ or a mixture thereof.

10. The method according to claim 8, characterized in that the fluorescent label is a fluorescent label for time-released fluorescence.

11. The method according to claim 10, characterized in that the fluorescent label is a complex, a micelle, or a chelate comprising a lanthanoid.

12. The method according to claim 11, characterized in that the lanthanoid is Eu³⁺ and/or Tb³⁺.

13. The method according to claim 8, characterized in that the fluorescent label is a fluorescent label for fluorescent energy transfer.

14. The method according to claim 13, characterized in that the fluorescent label is fluorescein:rhodamine.

15. The method according to claim 5, characterized in that the detectable label is a luminescent label.

16. The method according to claim 15, characterized in that the luminescent label is a luminescent label for chemiluminescence.

17. The method according to claim 16, characterized in that the luminescent label is an (iso-)luminol derivative and/or an acridine ester.

18. The method according to claim 15, characterized in that the luminescent label is a luminescent label for electroluminescence.

19. The method according to claim 18, characterized in that the luminescent label is an Ru²⁺-(2,2′-bipyridyl)₃ complex.

20. The method according to claim 15, characterized in that the luminescent label is a luminescent label for luminescent energy transfer.

21. The method according to claim 15, characterized in that the luminescent label is rhodamine:luminol.

22. The method according to claim 5, characterized in that the detectable label is a metal label.

23. The method according to claim 22, characterized in that the detectable label is a metal-labelled antibody.

24. The method according to claim 23, characterized in that the detectable label is selected from the group consisting of Au-labelled antibodies and Ag-labelled antibodies.

25. The method according to claim 5, characterized in that the detectable label is an enzyme label.

26. The method according to claim 25, characterized in that the enzyme label is an enzyme label for direct enzyme coupling.

27. The method according to claim 26, characterized in that the enzyme label is selected from the group consisting of alkaline phosphatase (AP), horseradish peroxidase (POD), microperoxidase, β-galactosidase, urease, glucose-oxidase, glucose-6-phosphate-dehydrogenase, hexokinase, bacterial luciferase and glow-worm luciferase or mixtures thereof.

28. The method according to claim 25, characterized in that the enzyme label is an enzyme label for enzyme substrate transfer.

29. The method according to claim 28, characterized in that the enzyme label is glucose-oxidase:horseradish peroxidase.

30. The method according to claim 25, characterized in that the enzyme label is an enzyme label for enzyme complementation.

31. The method according to claim 30, characterized in that the enzyme label is an inactive β-galactosidase:a-peptide.

32. The method according to claim 5, characterized in that the detectable label is a polymeric label.

33. The method according to claim 5, characterized in that the detectable label is selected from the group of latex dye particles and polyethyleneimine.

34. The method according to claim 1, characterized in that the addition of non-labelled substance in step b) is accomplished prior to the end of the biomolecule labelling reaction.

35. The method according to claim 34, characterized in that the addition of non-labelled substance in step b) is accomplished in the last third of the biomolecule labelling reaction.

36. The method according to claim 35, characterized in that the addition of non-labelled substance in step b) is accomplished immediately prior to the end of the biomolecule labelling reaction.

37. The method according to claim 1, characterized in that the non-labelled substance is a substance which is chemically, physically, or structurally related to the labelling substance.

38. The method according to claim 37, characterized in that the non-labelled substance is a non-labelled derivative of the labelling substance.

39. The method according to claim 37, characterized in that the non-labelled substance is identical to the utilized labelling substance apart from the actual labelling.

40. The method according to claim 1, characterized in that the separation of the labelled biomolecule is performed by any suitable purification process.

41. The method according to claim 40, characterized in that the purification process is a chromatographic process.

42. The method according to claim 5, characterized in that the labelling substance comprises a detectable label covalently bound to a carrier, said labelling substance being further covalently bound to or non-covalently associated with the biomolecule which is to be labelled.

43. The method according to claim 1, characterized in that the biomolecule labelling reaction is catalyzed by DNA-polymerase, RNA-polymerase, or reverse transcriptase.

44. The method according to claim 1, characterized in that the concentration ratio of the non-labelled substance to the labelling substance in the reaction mixture formed in step b) is in the range from 1:1 to 1000:1.

45. The method according to claim 44, characterized in that the concentration ratio of the non-labelled substance to the labelling substance in the reaction mixture formed in step b) is in the range from 10:1 to 100:1.
